(19) United States
(12) Patent Application Publication
(10) Pub. No.: US 2024/0180890 A1
(43) Pub. Date: Jun. 6, 2024

(54) VACCINE ADJUVANT

(71) Applicant: Celleron Therapeutics Limited, Oxford (GB)

(72) Inventor: Nicholas La Thangue, Oxford (GB)

(21) Appl. No.: 18/283,158

(22) PCT Filed: Mar. 31, 2021
(86) PCT No.: PCT/GB21/50811

§ 371 Date: Sep. 20, 2023

(51) Int. Cl. A61K 31/454; A61K 39/00; A61K 39/39; A61P 37/04

(52) U.S. Cl. CPC A61K 31/454; A61K 39/39; A61P 37/04; A61K 2039/545; A61K 2039/55511

(57) ABSTRACT

The present invention relates to the therapeutic use of the HDAC inhibitor compound, N-(2-aminophenyl)-4-(1-[(1,3-dimethyl-1H-pyrazol-4-yl)methyl]piperidin-4-yl)benzamide, or a pharmaceutically acceptable salt or solvate thereof, as a vaccine adjuvant. The present invention also relates to the combination of N-(2-aminophenyl)-4-(1-[(1,3-dimethyl-1H-pyrazol-4-yl)methyl]piperidin-4-yl)benzamide and a vaccine and the therapeutic uses thereof.

### FIELD OF THE INVENTION

The present invention relates to the therapeutic use of the HDAC inhibitor compound, N-(2-aminophenyl)-4-(1-[(1,3-dimethyl-1H-pyrazol-4-yl)methyl]piperidin-4-yl)benzamide, or a pharmaceutically acceptable salt or solvate thereof, as a vaccine adjuvant. The present invention also relates to the combination of N-(2-aminophenyl)-4-(1-[(1,3-dimethyl-1H-pyrazol-4-yl)methyl]piperidin-4-yl)benzamide and a vaccine and the therapeutic uses thereof.

### BACKGROUND OF THE INVENTION

Vaccination programmes can be extremely effective at controlling the spread of dangerous and life-threatening pathogens. In some cases, world-wide vaccination programmes have led to the virtual elimination of some dangerous pathogens.

A vaccine is a biological preparation typically containing either a harmless/attenuated form of a known pathogen (e.g. a bacteria or virus), a particular antigenic component of the pathogen concerned, or a nucleic acid (RNA or DNA) vector that can cause cells in the subject's body to produce the antigenic component of the pathogen concerned. Upon administration of a vaccine to a subject, an immune response is triggered and antibodies are produced to bind to and promote the elimination of the harmless form of the pathogen or the antigenic component of the pathogen that is provided by the vaccine. The stimulation of the immune system in this way results in immunological memory in the vaccinated subject. This enables the subject's immune system to quickly react to, and eliminate, the pathogen if it ever enters their body in the future. The immunological memory can last for years and can provide long-term immunity to the pathogen concerned.

Vaccines are also now being exploited to target tumours by promoting immune responses to tumour-specific antigens or tumour-associated antigens. This has the ability to use the immune system to target and eliminate tumour cells bearing the tumour-specific antigens or tumour-associated antigens.

For a vaccine to be effective, there needs to be a sufficient immune response in the subject upon administration of the vaccine. For certain subjects, the immune response may not be sufficient in order for the vaccine to have its intended effect. For example, subjects who are immuno-compromised, such as subjects who are elderly, fighting infections, suffering diseases that affect the immune system (e.g. HIV or AIDS), undergoing chemotherapy, suffering from a genetically inherited disease (e.g. Wiskott-Aldrich disease or chronic garanulomatosis disease), and/or taking immuno-suppressant drugs (e.g., organ transplant patients).

Vaccine adjuvants are known in the art to accelerate, prolong, and/or enhance the quality of the specific immune response upon administration of vaccine. However, existing vaccine adjuvants may not be able to be used with certain vaccines and/or may not provide the desired enhancement of the immune response in subjects that is required, especially in certain subjects such as immuno-compromised subjects.

Despite vaccine adjuvants being known in the art there remains a need for new vaccine adjuvants that provide enhanced immune responses in a subject upon administration with a vaccine.

The present invention was devised with the foregoing in mind.

### SUMMARY OF THE INVENTION

Data is presented in the example section herein that shows that the HDAC inhibitor compound N-(2-aminophenyl)-4-(1-[(1,3-dimethyl-1H-pyrazol-4-yl)methyl]piperidin-4-yl)benzamide (CXD101; zabinostat) potentiates the immune response to an immunogenic protein in normal healthy cells and animals. CXD101/zabinostat is therefore exhibiting the properties of an immune sensitiser and has the potential to act as a vaccine adjuvant.

Thus, the present invention relates to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use as a vaccine adjuvant.

The present invention also relates to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use as an immune sensitiser.

The present invention also relates to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use as an immune potentiating agent.

The present invention also relates to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in a method of potentiating an immune response in a subject in need thereof, the method comprising administering an effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

The present invention also relates to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in a method of potentiating an immune response in a subject in need thereof, wherein N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered in combination with a therapeutically effective amount of a vaccine.

The present invention also relates to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in a method of sensitising the immune system of a subject, the method comprising administering an effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

The present invention also relates to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in a method of sensitising the immune system of a subject, wherein N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered in combination with a therapeutically effective amount of a vaccine.

The present invention also relates to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in the treatment or prevention of disease, disorder or condition to which an immune response is required, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered to a subject receiving vaccine therapy.

The present invention also relates to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in the treatment or prevention of disease, disorder or condition to which an immune response is required, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered in combination with a therapeutically effective amount of a vaccine.

The present invention also relates to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in the treatment or prevention of disease, disorder or condition to which an immune response is required, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is for simultaneous, separate or sequential administration with a therapeutically effective amount of a vaccine.

The present also relates to a method of treating or preventing a disease, disorder, condition to which an immune response is required, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

In another aspect, the present invention relates to a method of treating or preventing a disease, disorder, condition to which an immune response is required, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

The present invention also relates to a method of potentiating the immune response in a subject, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

In another aspect, the present invention relates to a method of potentiating the immune response to a vaccine in a subject, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

In another aspect, the present invention relates to a method of potentiating the effect of a vaccine, the method comprising administering to a subject in need thereof a therapeutically effective amount of a vaccine and a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof.

The present invention also relates to a method of sensitising the immune system of a subject, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

In another aspect, the present invention relates to a method of sensitising the immune system of a subject, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

In another aspect, the present invention relates to the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use as a vaccine adjuvant.

In another aspect, the present invention relates to the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use as an immune-sensitiser.

In another aspect, the present invention relates to the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use as an immune-potentiating agent.

In another aspect, the present invention relates to the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use in the treatment a disease, disorder or condition to which an immune response is required.

In another aspect, the present invention relates to the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for potentiating the immune response in a subject.

In another aspect, the present invention relates to the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for potentiating the immune response in a subject receiving vaccine therapy.

In another aspect, the present invention relates to the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for sensitizing the immune system of a subject.

In another aspect, the present invention relates to the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for sensitizing the immune system of a subject receiving vaccine therapy.

The present invention also relates to a combination comprising N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a vaccine.

In another aspect the present invention relates to a pharmaceutical product comprising a combination as defined herein.

In another aspect, the present invention relates to a pharmaceutical composition comprising a combination as defined herein, and one or more pharmaceutically acceptable excipients.

In another aspect, the present invention relates to a combination as defined herein, or a pharmaceutical product as defined herein, or a pharmaceutical composition as defined herein for use in therapy.

In another aspect, the present invention relates to a combination as defined herein, or a pharmaceutical product as defined herein, or a pharmaceutical composition as defined herein for use in the treatment or prevention of a disease, disorder or condition to which an immune response is required in a subject.

In another aspect, the present invention relates to a combination as defined herein, or a pharmaceutical product as defined herein, or a pharmaceutical composition as defined herein for use in potentiating the immune response in a subject.

In another aspect, the present invention relates to a combination as defined herein, or a pharmaceutical product as defined herein, or a pharmaceutical composition as defined herein for use sensitising the immune system of a subject.

In another aspect, the present invention relates to a method of potentiating an immune response in a subject, the method comprising administering a therapeutically effective amount of a combination as defined herein, a therapeutically effective amount of the pharmaceutical product as defined herein or a therapeutically effective amount of the pharmaceutical composition as defined herein to a subject in need thereof.

In another aspect, the present invention relates to a method of potentiating the immune response to a vaccine in a subject, the method comprising administering a therapeutically effective amount of a combination as defined herein, a therapeutically effective amount of the pharmaceutical product as defined herein or a therapeutically effective amount of the pharmaceutical composition as defined herein to a subject in need thereof.

In another aspect, the present invention relates to a method of sensitising the immune system of a subject, the method comprising administering a therapeutically effective amount of a combination as defined herein, a therapeutically effective amount of the pharmaceutical product as defined herein or a therapeutically effective amount of the pharmaceutical composition as defined herein to a subject in need thereof.

In another aspect, the present invention relates to a method of preventing or treating a disease, disorder or condition to which an immune response is required in a subject, the method comprising administering a therapeutically effective amount of a combination as defined herein, a therapeutically effective amount of the pharmaceutical product as defined herein or a therapeutically effective amount of the pharmaceutical composition as defined herein to a subject in need thereof.

In another aspect, the present invention relates to a use of a combination as defined herein in the manufacture of a medicament for treating or preventing a disease, disorder or condition to which an immune response is required in a subject.

In another aspect, the present invention relates to a use of a combination as defined herein in the manufacture of a medicament for sensitising the immune response in a subject.

In another aspect, the present invention relates to a use of a combination as defined herein in the manufacture of a medicament for potentiating the immune response in a subject.

Suitably, the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered simultaneously, sequentially or separately with the vaccine.

In another aspect, the present invention relates to a vaccine for use in the treatment or prevention of a disease, condition or disorder to which an immune response is required, wherein the vaccine is for simultaneous, separate or sequential administration with N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof.

Preferred, suitable, and optional features of any one particular aspect of the present invention described herein are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

References to “a pharmaceutically acceptable salt” of N-(2-aminophenyl)-4-(1-[(1,3-dimethyl-1H-pyrazol-4-yl)methyl]piperidin-4-yl)benzamide (CXD101/zabinostat), is refers to any salt form suitable for pharmaceutical use. Examples of pharmaceutically acceptable salts include an acid-addition salt of N-(2-aminophenyl)-4-(1-[(1,3-dimethyl-1H-pyrazol-4-yl)methyl]piperidin-4-yl)benzamide (CXD101/zabinostat), for example, an acid-addition salt with, for example, an inorganic or organic acid, for example hydrochloric, hydrobromic, sulfuric, phosphoric, trifluoracetic, formic, citric methane sulfonate or maleic acid.

References herein to N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, include, where appropriate, any isomeric, tautomeric, polymorphic, amorphous and solvate (e.g. hydrate) forms of the inhibitors. N-(2-aminophenyl)-4-(1-[(1,3-dimethyl-1H-pyrazol-4-yl)methyl]piperidin-4-yl)benzamide (CXD101/zabinostat), may also be administered in the form of a prodrug which is broken down in the human or animal body to release the active inhibitor. Examples of pro-drugs include in vivo cleavable ester derivatives of the inhibitors that may be formed at a carboxy group or a hydroxy group in an inhibitor compound and in-vivo cleavable amide derivatives that may be formed at a carboxy group or an amino group in an inhibitor compound. Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

References herein to the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, being administered “in combination with” a vaccine, or vice versa, unless otherwise stated otherwise, include the agents being administered sequentially, separately or simultaneously with one another.

As used herein “simultaneous administration” refers to therapy in which the both agents (e.g. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and vaccine) are administered at the same time, suitably as a mono-therapy.

As used herein “sequential administration” means that one agent is administered after the other, however, the time period between the administration of each agent is such that both agents are capable of acting therapeutically concurrently. Thus, administration “sequentially” may permit one agent to be administered within seconds, minutes, or a matter of hours after the other provided the circulatory half-life of the first administered agent is such that they are both concurrently present in therapeutically effective amounts. The time delay between the administration of the agents may vary depending on the exact nature of the agents, the interaction there between, and their respective half-lives.

As used herein, “separate administration” means that one agent is administered after the other, however, the time period between administration is such that the first administered agent is no longer present a therapeutically effective amount when the second agent is administered. Accordingly, the two agents exert their therapeutic effects separately. Nevertheless, the overall therapeutic effect observed when the two agents separately act therapeutically may be greater than either agent used alone.

As used herein the, “subject(s)” and/or “patient(s)”, suitably refer to mammals (e.g. humans and non-human mammals such as livestock (cows, sheep, goats) or companion animals (cats, dogs, horses, rabbits). Suitably, the subject(s) and/or patient(s) are human(s).

As used herein, a “pharmaceutical product” refers to a product comprising a pharmaceutical. For instance, examples of a pharmaceutical product include a medical device, a pharmaceutical composition and a kit of parts suitably comprising one or more devices, containers and/or pharmaceuticals.

As used herein, the term “potentiate,” means that any of the mentioned activities of the compound embodied herein, are, e.g., increased, enhanced, promoted. Potentiation can increase activity more than 1-fold, 2-fold, 3-fold, 5-fold, 10-fold, 100-fold, etc., over baseline values.

### Therapeutic uses of N-(2-aminophenyl)-4-(1-[(1,3-dimethyl-1H-pyrazol-4-yl)methyl]piperidin-4-yl)benzamide

The present invention resides in the recognition that N-(2-aminophenyl)-4-(1-[(1,3-dimethyl-1H-pyrazol-4-yl)methyl]piperidin-4-yl)benzamide (CXD101/zabinostat), stimulates an immune response in subjects, as shown in the Examples. Without wishing to be bound by any theory, N-(2-aminophenyl)-4-(1-[(1,3-dimethyl-1H-pyrazol-4-yl)methyl]piperidin-4-yl)benzamide (CXD101/zabinostat) augments the expression of MHC (major histocompatibility complex) in cells and/or augments the response of T-cells upon presentation of an antigen. Therefore, N-(2-aminophenyl)-4-(1-[(1,3-dimethyl-1H-pyrazol-4-yl)methyl]piperidin-4-yl)benzamide (CXD101/zabinostat) is particularly well suited as an adjuvant, in particular a vaccine adjuvant.

In one aspect, there is N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use as a vaccine adjuvant.

Suitably, there is provided N-(2-aminophenyl)-4-(1-[(1,3-dimethyl-1H-pyrazol-4-yl)methyl]piperidin-4-yl)benzamide, or a pharmaceutically acceptable salt or solvate thereof, for use as an immune potentiating agent.

Suitably, there is provided N-(2-aminophenyl)-4-(1-[(1,3-dimethyl-1H-pyrazol-4-yl)methyl]piperidin-4-yl)benzamide, or a pharmaceutically acceptable salt or solvate thereof, for use as an immune sensitising agent.

In one embodiment, there is provided N-(2-aminophenyl)-4-(1-[(1,3-dimethyl-1H-pyrazol-4-yl)methyl]piperidin-4-yl)benzamide, or a pharmaceutically acceptable salt or solvate thereof, for use in a method potentiating an immune response in a subject in need thereof, the method comprising administering an effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy. In one embodiment, the immune response is induced or enhanced. In one embodiment, the immune response in the subject is more than 1-fold, such as 2-fold, 3-fold, 5-fold, or 10-fold over the baseline value. The baseline value refers to the immune response in the subject wherein the subject soley receives vaccine therapy, i.e., the subject has not received N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof.

In one embodiment, the subject is susceptible to or may have a disease, disorder or condition to which an immune response is required. In one embodiment, the subject is susceptible to or may have a pre-cancerous condition, cancer, an infectious disease, or an allergy. In one embodiment, the subject is susceptible to or may have an infectious disease or an allergy.

Suitably, there is provided N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in the treatment or prevention of a disease, disorder or condition to which an immune response is required, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered to a subject receiving vaccine therapy.

In one embodiment, the disease, disorder or condition to which an immune response is required is selected from a pre-cancerous condition, cancer, an infectious disease, or an allergy. In one embodiment, the disease, disorder or condition to which an immune response is required is selected from an infectious disease or an allergy.

In one aspect, there is provided a method of treating or preventing a disease, disorder, condition to which an immune response is required, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

Suitably, there is provided a method of potentiating an immune response, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

Suitably, there is provided a method of sensitising the immune system of a subject in need thereof, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

In another aspect, the present invention relates to a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in the manufacture of medicament for use as a vaccine adjuvant.

Suitably, the present invention relates to a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in the manufacture of medicament for use as an immune-sensitiser.

Suitably, the present invention relates to a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in the manufacture of medicament for use as an immune potentiating agent.

In one embodiment, the medicament is adminstered to a subject in combination with a therapeutically effective amount of a vaccine. In one embodiment, the medicament and the vaccine are adminstered simultaneously, sequentially or separately.

In another aspect, the present invention relates to a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in the manufacture of medicament for treating or preventing a disease, disorder, condition to which an immune response is required.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered to a subject in combination with a vaccine. In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered simultaneously, separately or sequentially with a vaccine.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered at a dosage of 0.5 mg to 40 mg per day optionally in a divided dose, 1 mg to 30 mg per day optionally in a divided dose, 1 mg to 20 mg per day optionally in a divided dose, or 1 mg to 10 mg per day optionally in a divided dose. Suitably, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered twice daily.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered on at least the same day as the vaccine. In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered on the same day as the vaccine. In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered for at least 7 days after administration of the vaccine. In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered on the same day as the vaccine and for at least 7 days after administration of the vaccine.

In one embodiment, the subject is immuno-comprised. Suitably, the subject is a subject who is undergoing chemotherapy, a subject who is taking an immuno-supressant drug, a subjectwith an infection, a subjectwho is suffering a disease that affects the immune system (e.g. AIDS or HIV), a subject suffering from a genetically inherited disease (e.g. Wiskott-Aldrich disease or chronic garanulomatosis disease), or a subject is elderly (for example, a subject is who at least 60 years old, such as at least 65, 70, 75, 80, 85, or 90 years old).

In one embodiment, the subject is receiving vaccine therapy.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, potentiates the immune response to a vaccine in the subject.

In one embodiment, the disease, disorder or condition to which an immune response is required is selected from a pre-cancerous condition, cancer, an infectious disease, or an allergy. In one embodiment, the disease, disorder or condition to which an immune response is required is selected from an infectious disease or an allergy.

In one aspect, there is provided N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in augmenting the expression of MHC in a subject.

In one aspect, there is provided the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in the manufacture of a medicament for use in augmenting the expression of MHC in a subject.

In one aspect, there is provided a method of augmenting the expression of MHC in a subject, the method comprising administering to a subject a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof.

In one aspect, there is provided N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in augmenting the T-cell immune response in a subject.

In one aspect, there is provided the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in the manufacture of a medicament for use in augmenting the T-cell immune response in a subject.

In one aspect, there is provided a method of augmenting the T-cell immune response in a subject, the method comprising administering to a subject a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof.

### Combination

The inventors have surprisingly discovered that the HDAC inhibitor compound, N-(2-aminophenyl)-4-(1-[(1,3-dimethyl-1H-pyrazol-4-yl)methyl]piperidin-4-yl)benzamide (CXD101/zabinostat) can significantly potentiate the therapeutic effects of a vaccine. This is especially useful for subjects who are immuno-compromised, for example and not limited to subjects who are elderly, undergoing chemotherapy, taking an immunosuppressant drug, suffering from a genetically inherited disease, or fighting an infection, wherein an immune response to a vaccine would otherwise not be to the desired level.

In an embodiment, the HDAC inhibitor compound, N-(2-aminophenyl)-4-(1-[(1,3-dimethyl-1H-pyrazol-4-yl)methyl]piperidin-4-yl)benzamide (CXD101/zabinostat) synergistically potentiates the therapeutic effect of a vaccine, thereby rendering the subject who was administered the vaccine and CDX101 with a greater immunity against the disease, condition or disorder they were vaccinated for.

Thus, the combination treatment of the present invention has the potential to provide better outcomes in subjects, especially immuno-compromised subjects.

The present invention resides in the recognition that the HDAC inhibitor compound, N-(2-aminophenyl)-4-(1-[(1,3-dimethyl-1H-pyrazol-4-yl)methyl]piperidin-4-yl)benzamide (CXD101/zabinostat), is particularly suited to use in combination with vaccines.

In one aspect, there is provided a combination comprising N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a vaccine.

In one aspect, there is provided N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in the treatment or prevention of a disease, disorder or condition to which an immune response is required, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered in combination with a therapeutically effective amount of a vaccine.

In one aspect, there is provided N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in sensitising the immune system of a subject, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered in combination with a therapeutically effective amount of a vaccine to a subject.

In one aspect, there is provided N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in potentiating the immune response of a vaccine in a subject, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered in combination with a therapeutically effective amount of a vaccine to a subject.

In one embodiment, there is provided N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in potentiating the immune response of a vaccine in a subject, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered in combination with a therapeutically effective amount of a vaccine to a subject, either sequentially, separately or simultaneously.

In one aspect, there is provided a method of treating or preventing a disease, disorder, condition to which an immune response is required, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

In one aspect, there is provided a method of potentiating the effect of a vaccine, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

In one aspect, there is provided a method of potentiating the immune response to a vaccine in a subject, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

In one embodiment, there is provided a method of potentiating the immune response to a vaccine in a subject, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine, either sequentially, separately or simultaneously.

In one aspect, there is provided a method of sensitising the immune system of a subject, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

In one aspect, there is provided a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use in the treatment a disease, disorder or condition to which an immune response is required, wherein the medicament is administered in combination with a vaccine.

In one aspect, there is provided a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use as a vaccine adjuvant, wherein the medicament is administered with a vaccine.

In one aspect, there is provided a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use as an immune-sensitiser, wherein the medicament is administered in combination with a vaccine.

In one aspect, there is provided a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use as an immune-potentiating agent, wherein the medicament is administered in combination with a vaccine.

Suitably, there is provided a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for potentiating the immune response to a vaccine in subject, wherein the medicament is administered in combination with a vaccine to the subject.

In one aspect, there is provided a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for potentiating the immune response to a vaccine in subject, wherein the medicament is administered in combination with a vaccine to the subject, either simultaneously, separately or sequentially with a vaccine.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered simultaneously, separately or sequentially with the vaccine.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, potentiates the immune response to a vaccine in the subject.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered at a dosage of 5 mg to 40 mg per day optionally in a divided dose, 1 mg to 30 mg per day optionally in a divided dose, 1 mg to 20 mg per day optionally in a divided dose, or 1 mg to 10 mg per day optionally in a divided dose. Suitably, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered twice daily.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered on at least the same day as the vaccine. In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered on the same day as the vaccine. In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered for at least 7 days after administration of the vaccine. In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered on the same day as the vaccine and for at least 7 days after administration of the vaccine.

An effective amount of a combination of an N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, or the vaccine for use in the combination therapy of the invention is an amount sufficient to treat or prevent a disorder, condition or disease referred to herein, slow its progression and/or reduce the symptoms associated with the condition.

The size of the dose for therapeutic or prophylactic purposes of a combination of the invention will naturally vary according to the nature and severity of the conditions, the age and sex of the animal or subject and the route of administration, according to well-known principles of medicine.

In using a combination of the invention for therapeutic or prophylactic purposes it will generally be administered with a therapeutically effective dose of the vaccine. These dosages are known in the art and will vary from one vaccine to another. The dosage may, for example, be in the range of 0.1 mg/kg to 30 mg/kg body weight. Suitable doing schedules are known in the art.

In one embodiment, the subject is immuno-comprised. Suitably, the subject is a subject who is undergoing chemotherapy, a subject who is taking an immuno-suppressant drug, a subject with an infection, a subject who is suffering a disease that affects the immune system (e.g. HIV or AIDS), a subject suffering from a genetically inherited disease (e.g. Wiskott-Aldrich disease or chronic garanulomatosis disease), or a subject is elderly (for example, a subject is who at least 60 years old, such as at least 65, 70, 75, 80, 85, or 90 years old).

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, potentiates the immune response to a vaccine in the subject.

In one embodiment, the disease, disorder or condition to which an immune response is required is selected from a pre-cancerous condition, cancer, an infectious disease, or an allergy. In one embodiment, the disease, disorder or condition to which an immune response is required is selected from an infectious disease or an allergy.

In one aspect, there is provided N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in augmenting the expression of MHC in a subject, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered in combination with a therapeutically effective amount of a vaccine.

In one aspect, there is provided the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in the manufacture of a medicament for use in augmenting the expression of MHC in a subject, wherein the medicament is administered in combination with a therapeutically effective amount of a vaccine.

In one aspect, there is provided a method of augmenting the expression of MHC in a subject, the method comprising administering to a subject a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

In one aspect, there is provided N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use in augmenting the T-cell immune response in a subject, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered in combination with a therapeutically effective amount of a vaccine.

In one aspect, there is provided the use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in the manufacture of a medicament for use in augmenting the T-cell immune response in a subject, wherein the medicament is administered in combination with a therapeutically effective amount of a vaccine.

In one aspect, there is provided a method of augmenting the T-cell immune response in a subject, the method comprising administering to a subject a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

In one embodiment, the potentiating effect of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, causes an enhanced or induced immune response. In one embodiment, the immune response in the subject is more than 1-fold, such as 2-fold, 3-fold, 5-fold, or 10-fold over the baseline value. The baseline value refers to the immune response in the subject wherein the subject soley receives vaccine therapy, i.e., the subject has not received N-(2-aminophenyl)-4-(1-[(1,3-dimethyl-1H-pyrazol-4-yl)methyl]piperidin-4-yl)benzamide.

### CXD101/Zabinostat

CXD101/zabinostat (previously known as AZD9468) is a Class I-selective histone deacetylase (HDAC) inhibitor with specificity for Class I isoforms HDAC1 (63 nM IC50), HDAC2 (570 nM IC50), and HDAC3 (550 nM IC50), and no activity (≥2500 nM) against HDAC Class II.

The chemical name of CXD101/zabinostat is N-(2-aminophenyl)-4-(1-[(1,3-dimethyl-1H-pyrazol-4-yl)methyl]piperidin-4yl)benzamide. It has a molecular weight of 403.52, and formula C24H29N5O. CXD101/zabinostat is a white or off-white crystalline solid. It is dibasic with a pKas of 3.2 and 9. It displays pH-dependent solubility, with a solubility of 0.5 mg/mL to >20 mg/mL across the pH range 1 to 8 at 25° C. The melting point of CXD101/zabinostat is approximately 172° C. The UV absorbance maxima are 199 and 229 nm.

The structure of CXD101/zabinostat is shown below:

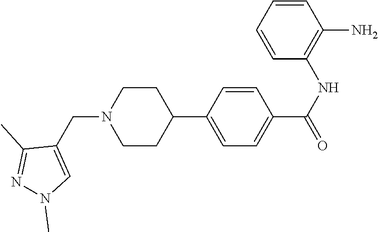 [SMILES: Cc1nn(C)cc1CN1CCC(c2ccc(C(=O)Nc3ccccc3N)cc2)CC1]

The CXD101/zabinostat compound is suitably administered orally, optionally in the form of a tablet or capsule (for example, a tablet or capsule containing 0.25 to 40 mg of CXD101/zabinostat, such as 0.5 mg to 30 mg, 0.5 to 20 mg, 0.5 to 10 mg, or 0.5 to 5 mg). Typically, the dosage for the CXD101/zabinostat compound will be 0.5-40 mg per day optionally in a divided dose, such as 1-30 mg per day optionally in a divided dose, 1-20 mg per day optionally in a divided dose, or 1-10 mg per day optionally in a divided dose. Suitably, the CXD101/zabinostat compound is adminstered twice daily.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, potentiates the immune response to a vaccine in the subject.

### Vaccines

Any vaccine may be used in the combination therapy defined herein.

In one embodiment, the vaccine is an allergen vaccine, an infectious disease vaccine, a mucosal vaccine or an anti-cancer vaccine.

In one embodiment, the vaccine is an allergen vaccine, an infectious disease vaccine, or a mucosal vaccine.

An allergen vaccine is a vaccine which is produced by producing an IgG antibody against an allergen by blocking the action of IgE causing allergy by administering an allergen to a living body or by blocking the action of IgE which is a cause of allergy in type 1 helper T cell Cells) and decreases type 2 helper T cells (Th2 cells) involved in allergic symptoms, and it can suppress allergic symptoms by hyposensitisation. Allergen vaccines consist of allergens that cause various allergies. Examples of the allergen to be used in combination with N-(2-aminophenyl)-4-(1-[(1,3-dimethyl-1H-pyrazol-4-yl)methyl]piperidin-4-yl)benzamide, or a pharmaceutically acceptable salt or solvate thereof, include, but are not limited to, food allergens, house dust allergens, pollen allergens such as cedar pollen, and allergens such as body hair of animals. Specifically, examples of pollen allergens include cedar pollen allergen (Cry j 1, Cry j 2), ragweed allergen (Amba 1, Amba 2, Amba 5, Ambt 5, Ambp 5), camomilea allergen (Dacg 2), etc. Examples of food allergens include casein, lactalbumin, lactoglobulin, ovomucoid, ovalbumin, conalbumin and the like. Examples of mite allergens include Derf 1, Derf 2, Zen 1, Derp 1, Derp 2 and the like.

In one embodiment, the infectious disease vaccine is a vaccine used to treat or prevent viral, bacterial, fungal, protozoa and/or parasite infections.

Vaccines for infectious diseases include inactivated complete vaccines, subunit vaccines, toxoids and the like. These vaccines cause subjects to be immunized against pathogens such as bacteria, viruses, Rickettsia, parasites.

In one embodiment, the infectious disease vaccine is a vaccine use to treat or prevent, in the case of a human being, for example, influenza, poliovirus, Japanese encephalitis, tubercle Bacillus, rabies, human papilloma virus, malaria parasite, SARS, typhoid fever, paratyphoid fever, plague, Salmonellosis, Staphylococcus aureus, and/or pertussis.

In one embodiment, the infectious disease vaccine is a vaccine use to treat or prevent, in the case of a non-human animal, for example, an equine influenza virus, equine herpes virus, equine meningitis virus, foot and mouth disease virus, Escherichia coli (cow mastitis), rabies, feline panleukopenia, cat rhinotracheitis, infectious bovine rhinotracheitis, PRRS virus (swine pneumonia), and/or Mycoplasma (pig pneumonia). A parasitic infection such as type 3 parainfluenza, bovine viral diarrhoea, bovine adenovirus, porcine parvovirus, canine adenovirus, canine distemper virus, canine parvovirus, canine parainfluenza, avian influenza, brucellosis, vibriosis, leptospirosis, and/or clostridial infection.

In one embodiment, the vaccine is used to prevent and/or treat adenovirus, anthrax, cholera, covid-19, diphtheria, hepatitis A, hepatitis B, Haemophilus influenzae type b (Hib), Human Papillomavirus (HPV), seasonal influenza, Japanese Encephalitis, Measles, Meningococcal, Mumps, Pertussis, Pneumococcal, Polio, Rabies, Rotavirus, Rubella, Shingles, Smallpox, Tetanus, Tuberculosis, Typhoid Fever, Varicella, and/or Yellow Fever in a subject.

In one embodiment, the vaccine is used to prevent and/or treat cancer by presenting one or more tumour-specific antigens or tumour-associated antigens to the subject's immune system. Tumour-specific antigens and tumour associated antigens are well known in the art and include alphafetoprotein (AFP; germ cell tumours and hepatocellular carcinoma), carcinoembryonic antigen (CEA; bowel cancers), CA-125 (ovarian cancer), MUC-1 (breast cancer), epithelial tumour antigen (ETA; breast cancer), tyrosinase (malignant melanoma), and/or melanoma-associated antigen (MAGE; malignant melanoma).

Suitably, the anti-cancer vaccine is a cell-based anti-cancer vaccine (for example, GVAX, gemogenovatucel-T (e.g. Vigil®), sipuleucel-T (e.g. Provenge®), MART-1).

Suitably, the anti-cancer vaccine is virus based anti-cancer vaccine (for example talimogene laherparepvec (TVEC), rilimogene galvacirepvec/rilimogene glafolivec (PROSTVAC), or TroVax (5T4 antigen)). Virus based vaccines can be either directly oncolytic or deliver the tumour antigen to augment the response.

Suitably, the anti-cancer vaccine is a neoantigen-targeted cancer vaccine (for example, SLATE, or GRANITE-001). Suitably, the neoantigen-targeted cancer vaccine comprises a synthetic long peptide, mRNA and/or a neoantigen expressed from a virus (for example adenovirus platform ChAdOx).

Suitably, the anti-cancer vaccine is an adoptive cell therapy vaccine. Suitably, the adoptive cell therapy vaccine is a tumour-infiltrating lymphocyte (TIL) therapy vaccine, engineered T Cell Receptor (TCR) therapy vaccine, chimeric antigen receptor (CAR) T cell therapy vaccine, or a natural killer (NK) cell therapy vaccine.

Suitably the anti-cancer vaccine is a dendritic cell vaccine or a nanoparticle-based vaccine

In one embodiment, the vaccine is used to prevent and/or treat more than one disease, condition or disorder in a subject, for example 2, 3 or more diseases, conditions or disorders (i.e., the measles, mumps and rubella vaccine). In one embodiment, the vaccine is a combination vaccine.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered simultaneously, separately or sequentially with a vaccine.

### Therapeutic Uses

In one aspect, the present invention relates to a combination as herein described, a pharmaceutical product as herein described, or a pharmaceutical composition as herein described for use in therapy.

In aspect, the present invention relates to a combination as herein described, a pharmaceutical product as herein described, or a pharmaceutical composition as herein described for potentiating an immune response in a subject.

Suitably, the present invention relates to a combination as herein described, a pharmaceutical product as herein described, or a pharmaceutical composition as herein described for potentiating an immune response to a vaccine in a subject.

In one aspect, the present invention relates to a combination as herein described, a pharmaceutical product as herein described, or a pharmaceutical composition as herein described for sensitising the immune system of a subject.

In one aspect, the present invention relates to a combination as herein described, a pharmaceutical product as herein described, or a pharmaceutical composition as herein described for use in the treatment or prevention of disease, disorder or condition to which an immune response is required in a subject.

In one embodiment, the present invention relates to a combination as herein described, a pharmaceutical product as herein described, or a pharmaceutical composition as herein described for use in the treatment or prevention of an allergy, an infectious disease, pre-cancerous condition, or cancer. In one embodiment, the present invention relates to a combination as herein described, a pharmaceutical product as herein described, or a pharmaceutical composition as herein described for use in the treatment or prevention of an allergy or an infectious disease.

In one embodiment, the present invention relates to a combination as herein described, a pharmaceutical product as herein described, or a pharmaceutical composition as herein described for use in the treatment or prevention of an allergy, or an infectious disease.

In one aspect, the present invention relates to a method of potentiating an immune response in a subject, the method comprising administering a therapeutically effective amount of the combination as described herein, a therapeutically effective amount of the pharmaceutical product as described herein, or a therapeutically effective amount of the pharmaceutical composition as described herein to the subject in need thereof.

Suitably, the present invention relates to a method of potentiating the immune response to a vaccine in a subject, the method comprising administering a therapeutically effective amount of the combination as described herein, a therapeutically effective amount of the pharmaceutical product as described herein, or a therapeutically effective amount of the pharmaceutical composition as described herein to the subject in need thereof.

In one aspect, the present invention relates to a method of sensitising the immune system of a subject, the method comprising administering a therapeutically effective amount of the combination as described herein, a therapeutically effective amount of the pharmaceutical product as described herein, or a therapeutically effective amount of the pharmaceutical composition as described herein to the subject in need thereof.

In one aspect, the present invention relates to a method of preventing or treating a disease, disorder or condition to which an immune response is required in a subject, the method comprising administering a therapeutically effective amount of the combination as described herein, a therapeutically effective amount of the pharmaceutical product as described herein, or a therapeutically effective amount of the pharmaceutical composition as described herein to the subject in need thereof.

In one embodiment, the present invention relates to a method of preventing or treating an allergy, an infectious disease, a pre-cancerous condition, or cancer, the method comprising administering a therapeutically effective amount of the combination as described herein, a therapeutically effective amount of the pharmaceutical product as described herein, or a therapeutically effective amount of the pharmaceutical composition as described herein to the subject in need thereof. In one embodiment, the present invention relates to a method of preventing or treating an allergy or an infectious disease, the method comprising administering a therapeutically effective amount of the combination as described herein, a therapeutically effective amount of the pharmaceutical product as described herein, or a therapeutically effective amount of the pharmaceutical composition as described herein to the subject in need thereof.

In one embodiment, the present invention relates to a method of preventing or treating an allergy or an infectious disease, the method comprising administering a therapeutically effective amount of the combination as described herein, a therapeutically effective amount of the pharmaceutical product as described herein, or a a therapeutically effective amount of pharmaceutical composition as described herein to the subject in need thereof.

In one aspect, there is provided a use of a combination as herein described in the manufacture of a medicament for potentiating an immune response in a subject.

Suitably, there is provided a use of a combination as herein described in the manufacture of a medicament for potentiating an immune response to a vaccine in a subject.

In one aspect, there is provided a use of a combination as herein described in the manufacture of a medicament for sensitisiting the immune system of a subject.

In one aspect, there is provided a use of a combination as herein described in the manufacture of a medicament for treating or preventing a disease, disorder or condition to which an immune response is required in a subject.

Suitably, there is provided a use of a combination as herein described in the manufacture of a medicament for use in the treatment or prevention of an allergy, an infectious disease, a pre-cancerous condition, or cancer in a subject. Suitably, there is provided a use of a combination as herein described in the manufacture of a medicament for use in the treatment or prevention of an allergy or an infectious disease in a subject.

In another aspect, the present invention relates to a vaccine, for use in the treatment or prevention of a disease, disorder or condition to which an immune response is required in a subject, wherein the vaccine is administered simultaneously, separately or sequentially with N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, as defined herein.

In another aspect, the present invention relates to a use of vaccine, in the manufacture of a medicament for treating or preventing a disease, disorder or condition to which an immune response is required in a subject, wherein the medicament is for simultaneous, separate or sequential administration with N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, as defined herein.

In another aspect, the present invention relates to a method of treating or preventing a disease, disorder or condition to which an immune response is required in a subject, the method comprising administering a vaccine simultaneously, separately or sequentially with N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, as defined herein.

In another aspect, the present invention relates to a method of potentiating the effect of a vaccine, the method comprising administering the vaccine separately, sequentially or simultaneously with N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, as defined herein.

In one aspect, there is provided a use of a combination as herein described in the manufacture of a medicament for use in the treatment or prevention of an allergy or an infectious disease in a subject.

In another aspect, the present invention relates to a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, treating or preventing a disease, disorder or condition to which an immune response is required in a subject, wherein the medicament is administered separately, sequentially or simultaneously with a vaccine.

In another aspect, the present invention relates to a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in the manufacture of medicament for potentiating the effect of a vaccine, wherein the medicament is administered separately, sequentially or simultaneously with a vaccine.

In another aspect, the present invention relates to a use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in the manufacture of medicament for sensitising the immune system, wherein the medicament is administered separately, sequentially or simultaneously with a vaccine.

In one embodiment, the disease, disorder or condition to which an immune response is required in a subject is selected from a pre-cancerous condition, cancer, an allergy or an infectious disease. In one embodiment, the disease, disorder or condition to which an immune response is required in a subject is selected from an allergy or an infectious disease.

In one embodiment, the infectious disease is a viral infectious disease, bacterial infectious disease, fungal infectious disease, protozoa infectious disease or parasite infectious disease.

In one embodiment, the allergy is a food allergy, a house dust allergy, a pollen allergy such as cedar pollen allergy, and an allergy to body hair of animals. Specifically, examples of pollen allergies include a cedar pollen allergy, a ragweed allergy, or a camomilea allergy. Examples of food allergies include an allergy to casein, lactalbumin, lactoglobulin, ovomucoid, ovalbumin, conalbumin and the like. Examples of mite allergies include an allergy to Derf 1, Derf 2, Zen 1, Derp 1, Derp 2 and the like.

In one embodiment, the infectious disease is, in the case of a human being, for example, influenza, poliovirus, Japanese encephalitis, tubercle Bacillus, rabies, human papilloma virus, malaria parasite, SARS, typhoid fever, paratyphoid fever, plague, Salmonellosis, Staphylococcus aureus, and/or pertussis.

In one embodiment, the infectious disease vaccine is, in the case of a non-human animal, for example, equine influenza virus, equine herpes virus, equine meningitis virus, foot and mouth disease virus, Escherichia coli (cow mastitis), rabies, feline panleukopenia, cat rhinotracheitis, infectious bovine rhinotracheitis, PRRS virus (swine pneumonia), and/or Mycoplasma (pig pneumonia). A parasitic infection such as type 3 parainfluenza, bovine viral diarrhoea, bovine adenovirus, porcine parvovirus, canine adenovirus, canine distemper virus, canine parvovirus, canine parainfluenza, avian influenza, brucellosis, vibriosis, leptospirosis, and/or clostridial infection.

In one embodiment, the disease, disorder or condition to which an immune response is required in a subject is adenovirus, anthrax, cholera, covid-19, diphtheria, hepatitis A, hepatitis B, Haemophilus influenzae type b (Hib), Human Papillomavirus (HPV), seasonal influenza, Japanese Encephalitis, Measles, Meningococcal, Mumps, Pertussis, Pneumococcal, Polio, Rabies, Rotavirus, Rubella, Shingles, Smallpox, Tetanus, Tuberculosis, Typhoid Fever, Varicella, and/or Yellow Fever in a subject.

In one embodiment, the disease, disorder or condition to which an immune response is required in a subject is cancer. Suitably, the cancer is selected from germ cell tumours, hepatocellular carcinoma, bowel cancers, ovarian cancer, breast cancer, malignant melanoma, prostate cancer, pancreatic cancer, melanoma, renal cell carcinoma, colorectal cancer, lung cancer, and/or solid tumour. Suitably, the anti-cancer vaccine is used to prevent and/or treat cancer by presenting one or more tumour-specific antigens or tumour-associated antigens to the subject's immune system. Tumour-specific antigens and tumour associated antigens are well known in the art and include alphafetoprotein (AFP; germ cell tumours and hepatocellular carcinoma), carcinoembryonic antigen (CEA; bowel cancers), CA-125 (ovarian cancer), MUC-1 (breast cancer), epithelial tumour antigen (ETA; breast cancer), tyrosinase (malignant melanoma), and/or melanoma-associated antigen (MAGE; malignant melanoma).

Suitably, the anti-cancer vaccine is a cell-based anti-cancer vaccine (for example, GVAX, gemogenovatucel-T (e.g. Vigil®), sipuleucel-T (e.g. Provenge®), MART-1).

Suitably, the anti-cancer vaccine is virus based-anti-cancer vaccine (for example talimogene laherparepvec (TVEC), rilimogene galvacirepvec/rilimogene glafolivec (PROSTVAC), or TroVax (5T4 antigen)). Virus based vaccines can be either directly oncolytic or deliver the tumour antigen to augment the response.

Suitably, the anti-cancer vaccine is a neoantigen-targeted cancer vaccine (for example, SLATE, or GRANITE-001). Suitably, the neoantigen-targeted cancer vaccine comprises a synthetic long peptide, mRNA and/or a neoantigen expressed from a virus (for example adenovirus platform ChAdOx).

Suitably, the anti-cancer vaccine is an adoptive cell therapy vaccine. Suitably, the adoptive cell therapy vaccine is a tumour-infiltrating lymphocyte (TIL) therapy vaccine, engineered T Cell Receptor (TCR) therapy vaccine, chimeric antigen receptor (CAR) T cell therapy vaccine, or a natural killer (NK) cell therapy vaccine.

Suitably the anti-cancer vaccine is a dendritic cell vaccine or a nanoparticle based vaccine.

In one embodiment, N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered simultaneously, separately or sequentially with a vaccine.

The combination therapy may be in the form of a combined formulation for simultaneous administration of the therapeutically active components or they may be administered as separate formulations. The separate formulations may be administered sequentially, separately or simultaneously.

In one embodiment the separate formulations of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, and a vaccine are administered simultaneously (optionally repeatedly).

In one embodiment the separate formulations of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, and a vaccine are administered sequentially (optionally repeatedly).

In one embodiment the separate formulations of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, and a vaccine are administered separately (optionally repeatedly).

The skilled person will understand that where the separate formulations of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, and a vaccine are administered sequentially or serially that this could be administration of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, followed by a vaccine or a vaccine followed by N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof.

Where the administration of the separate formulations of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a vaccine is sequential or separate, the delay in administering the second formulation should not be such as to lose the beneficial effect of the combination therapy.

In one embodiment, the subject is immuno-compromised. Suitably, the subject is a subject who is undergoing chemotherapy, a subject who is taking an immuno-suppressant drug, a subject with an infection, a subject who is suffering a disease that affects the immune system (e.g. AIDS or HIV), a subject suffering from a genetically inherited disease (e.g. Wiskott-Aldrich disease or chronic garanulomatosis disease), or a subject is elderly (for example, a subject is who at least 60 years old, such as at least 65, 70, 75, 80, 85, or 90 years old).

### Pharmaceutical Compositions

In one aspect the present invention relates to a pharmaceutical composition comprising a combination of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and an antigen.

Suitably, the pharmaceutical composition is in a form suitable for administration by injection, for example for oral, subcutaneous, intramuscular, intradermal, or in a form suitable for intranasal administration.

The pharmaceutical compositions may be obtained by conventional procedures using conventional pharmaceutical excipients well known in the art.

The amount of active ingredient that is combined with one or more excipients to produce a single dosage form will necessarily vary depending upon the individual treated and the particular route of administration.

### Routes of Administration

N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof may be administered to a subject by any appropriate or convenient route of administration, whether systemically/peripherally or topically (i.e., at the site of desired action).

Routes of administration for CDX101 include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

CXD101/zabinostat is suitably administered orally.

Suitably, routes of administration for the vaccine include oral, subcutaneous, intramuscular, intradermal, or intranasal.

Combinations with Additional Therapeutic Agents

The combination treatment defined herein may be applied as a sole therapy for the treatment of the specified condition or it may involve, in addition to the combination therapy of the present invention, one or more additional therapies (including treatment with another therapeutic agent, surgery or other therapeutic interventions).

Typically, the other therapeutic agent used in combination with the combination therapy of the present invention will be one or more therapeutic agents used as the standard of care for the treatment or prevention of the disease, condition, or disorder concerned. The other therapeutic agent may include, for example, another drug used for the treatment of the condition concerned, or an agent that modulates the biological response to the combination therapy of the invention.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

### Pharmaceutical Products

In another aspect, the present invention provides a pharmaceutical product comprising a combination of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, and a vaccine.

In one embodiment, the pharmaceutical product may comprise a kit of parts comprising separate formulations of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a vaccine. The separate formulations of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and the vaccine, may be administered sequentially, separately and/or simultaneously.

In another embodiment the pharmaceutical product is a kit of parts which comprises: a first container comprising N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, in association with a pharmaceutically acceptable adjuvant, diluent or carrier; and
a second container comprising a vaccine; and
a container means for containing said first and second containers.

In one embodiment, the pharmaceutical product may comprise a one or more unit dosage forms (e.g. vials, tablets or capsules in a blister pack). In one embodiment, one unit dose comprises N-(2-aminophenyl)-4-(1-[(1,3-dimethyl-1H-pyrazol-4-yl)methyl]piperidin-4-yl)benzamide (CXD101/zabinostat) compound and and the other unit dosage form is the vaccine. In another embodiment, the unit dosage form comprises both the N-(2-aminophenyl)-4-(1-[(1,3-dimethyl-1H-pyrazol-4-yl)methyl]piperidin-4-yl)benzamide (CXD101/zabinostat) compound and the vaccine (or antigenic component of the vaccine).

In one embodiment, the pharmaceutical product is a pharmaceutical composition.

Suitably, the CXD101/zabinostat compound is administered orally.

In one embodiment the pharmaceutical product or kit of parts further comprises means for facilitating compliance with a dosage regimen, for instance instructions detailing how to administer the combination.

### DESCRIPTION OF DRAWINGS

Embodiments of the invention will be described, by way of example only, with reference to the accompanying drawings, in which:

FIG. 1: Expression of MHC class I and class II genes in HCT116

Quantitative reverse transcription PCR (qRT-PCR) of MHC class I and class II genes in HCT116 cells treated for 3 days with 1, 2.5, 5 μM CXD101 or DMSO control; n=3; The acetylation mark (H3AcK14) was detected by immunoblotting.

FIG. 2: Expression of MHC class I and class II genes in A549 (high dose)

Quantitative reverse transcription PCR (qRT-PCR) of MHC class I and class II genes in A549 cells treated for 3 days with 1, 2.5, 5 μM CXD101 or DMSO control; n=3; The acetylation mark (H3AcK14) was detected by immunoblotting.

FIG. 3: Expression of MHC class I and class II genes in A549 (low dose)

Quantitative reverse transcription PCR (qRT-PCR) of MHC class I and class II genes in A549 cells treated for 3 days with 1, 10, 100, 1000 nM CXD101 or DMSO control; n=3; The acetylation mark (H3AcK14) was detected by immunoblotting.

FIG. 4: Expression of MHC class I and class II genes in BEAS-2B

Quantitative reverse transcription PCR (qRT-PCR) of MHC class I and class II genes in BEAS-2B cells treated for 3 days with 1, 10, 100, 1000 nM CXD101 or DMSO control; n=3; The acetylation mark (H3AcK14) was detected by immunoblotting.

FIG. 5: Expression of MHC class I and class II genes in NBE1

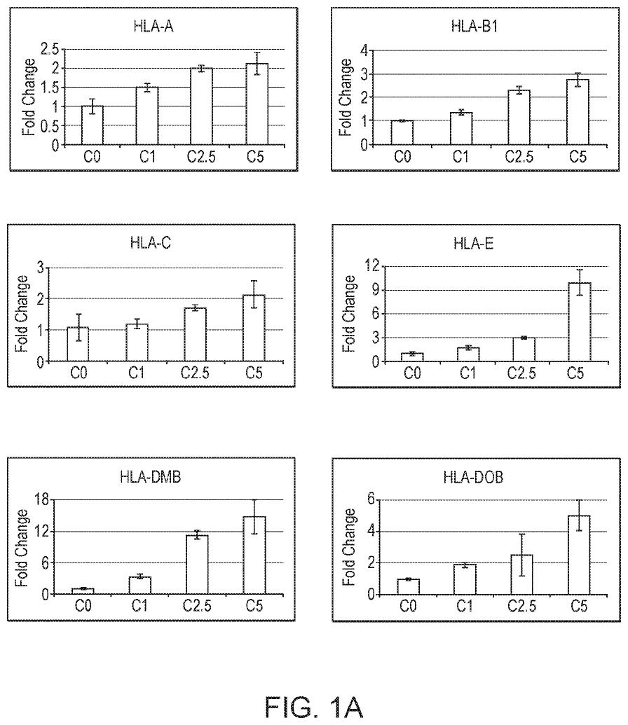

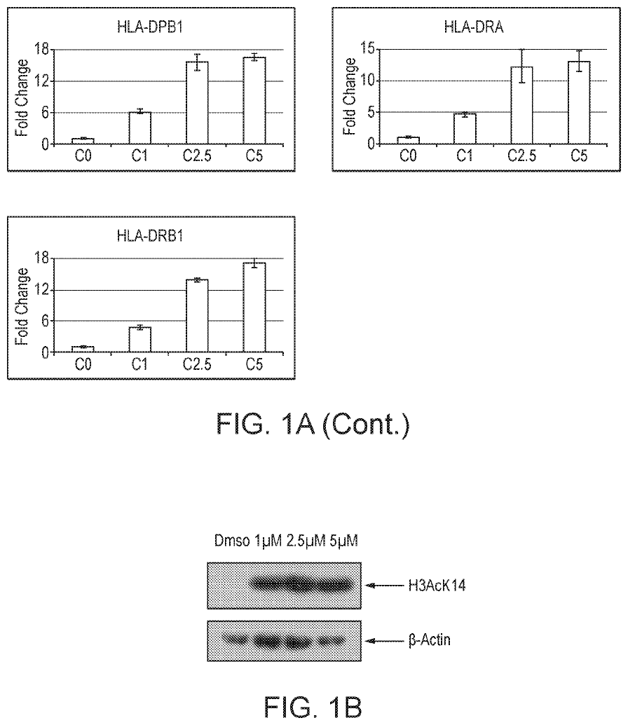

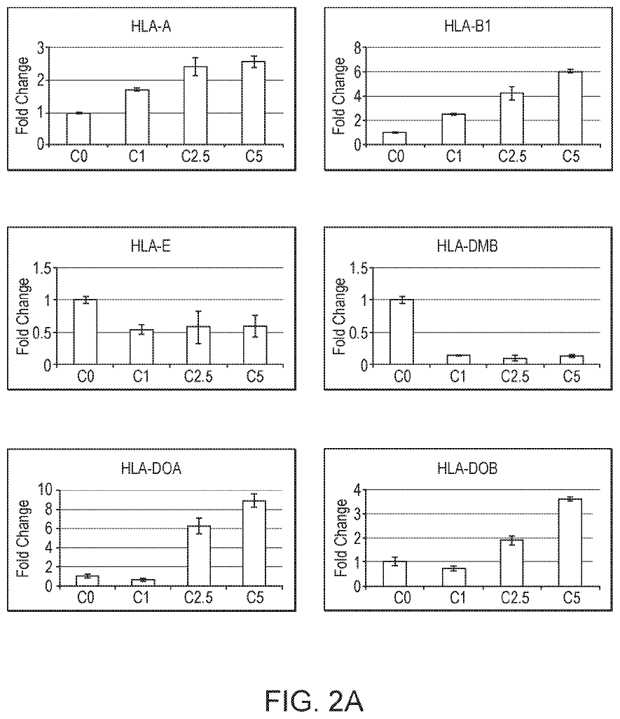

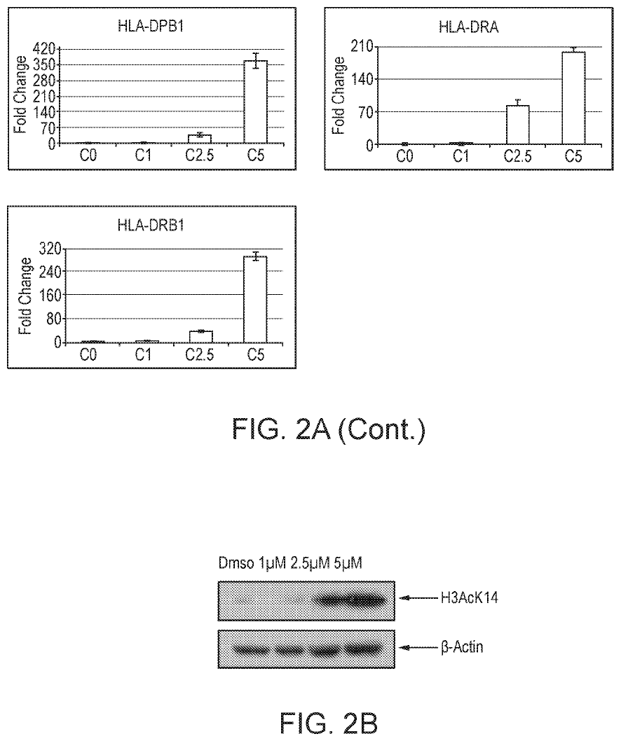

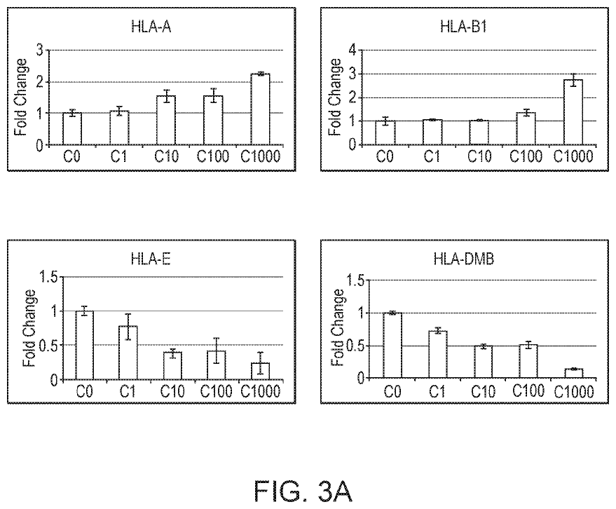

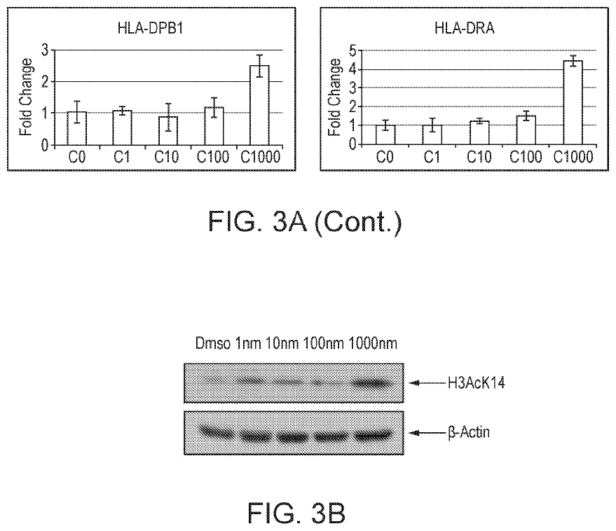

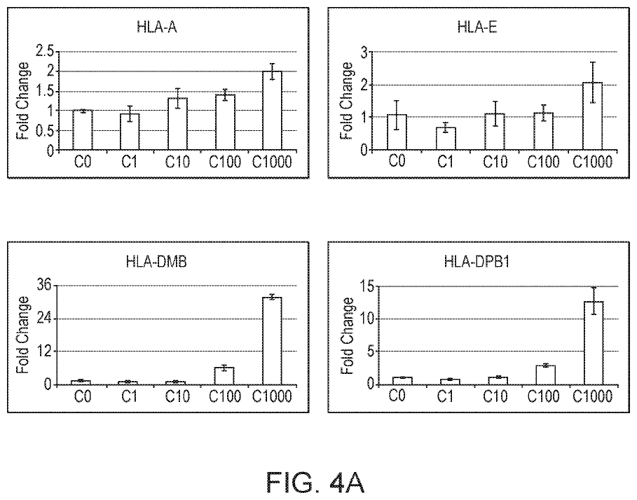

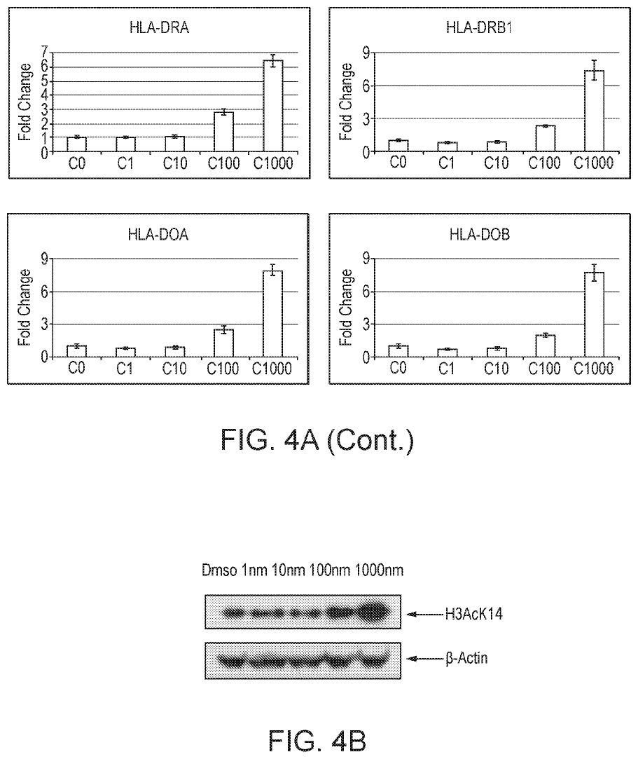

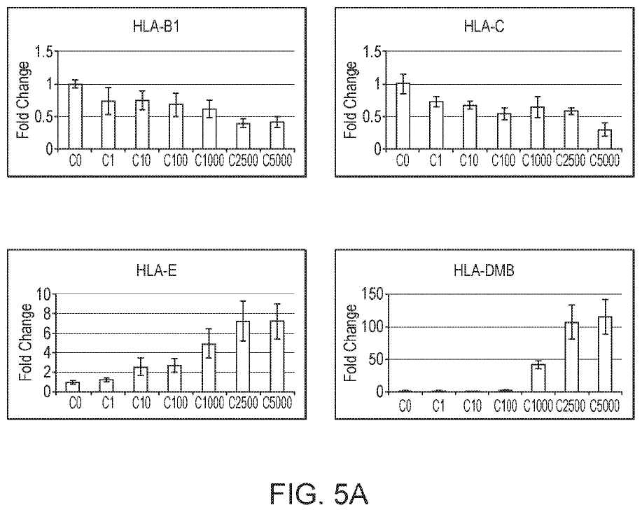

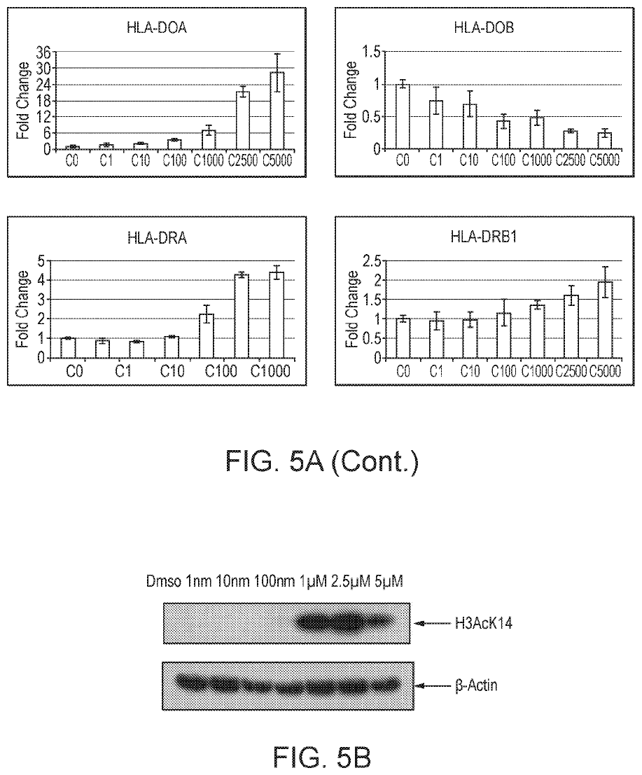

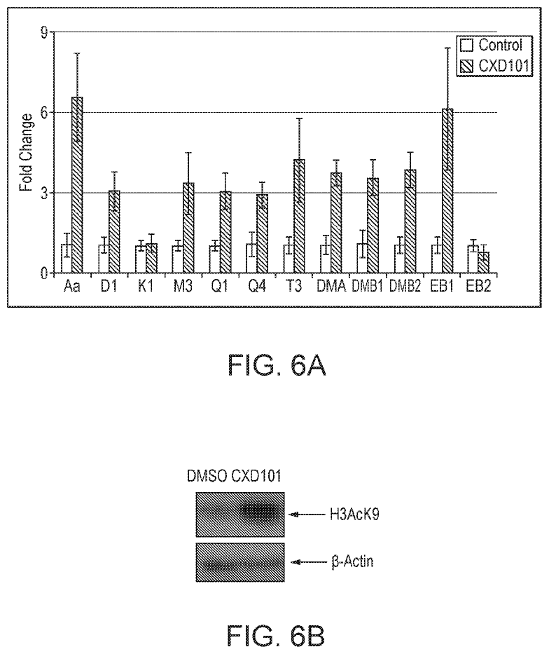

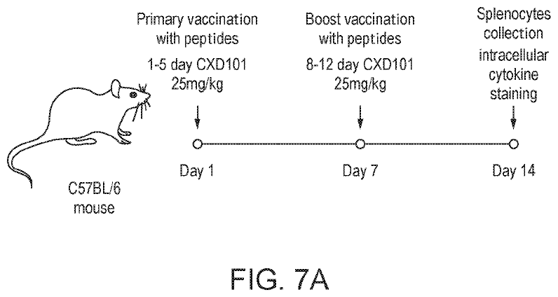

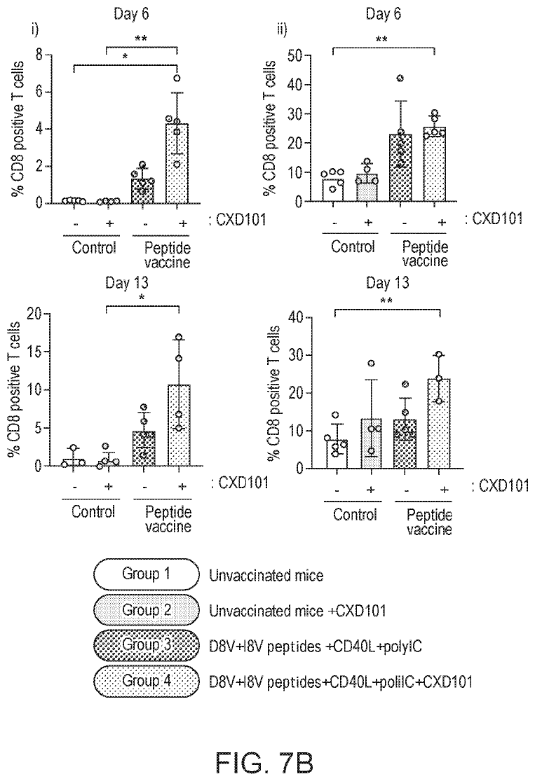

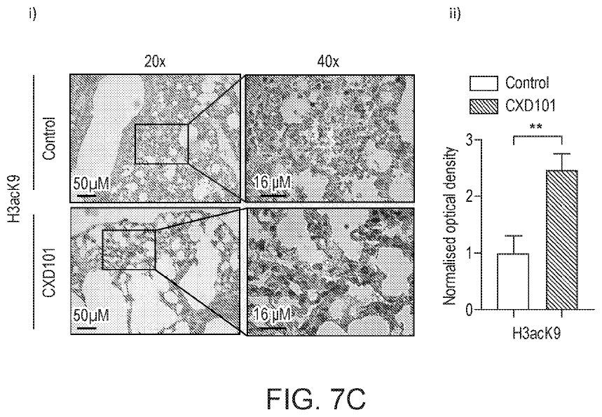

Quantitative reverse transcription PCR (qRT-PCR) of MHC class I and class II genes in NBE1 cells treated for 3 days with 1, 10, 100, 1000, 2500, 5000 nM CXD101 or DMSO control; n=3; The acetylation mark (H3AcK14) was detected by immunoblotting.

FIG. 6: Expression of MHC class I and class II genes in mouse lung tissue
A) Quantitative reverse transcription PCR (qRT-PCR) of MHC class I and class II genes in lung RNA from Balb/c mice treated for 14 days with 10 mg/kg CXD101 or DMSO control; n=4.
B) The acetylation mark (H3AcK9) was detected by immunoblotting.

FIG. 7: CXD101 induces immunogenicity against a peptide-based vaccine.
A) Schematic representation of the immunogenicity experiment with CXD101 and peptide vaccine. C57BL/6 mice were treated with orally administrated CXD101 at 10 mg/kg for 14 days (5 days on/2 days off) and intravenous D8V and 18V peptides vaccination (days 1 and 7) with respect to vehicle only control; n=5 per group;
B) 18V-peptide specific CD8 positive T cells with tetramers (i) and general T cell activation (CD8+ CD44+ CD62L− tetramer positive T cells) measurements by flow cytometry; n=5;
C) Representative examples of immunohistochemical staining of H3AcK9 in lungs (i) collected from C57BL/6 mice at 14 days treated with 25 mg/kg CXD101 and non-treated control. Original magnification: 20×, scale bar, 50 μm; and 63×; scale bar, 16 μm. n=3; (ii) results were quantified by ImageJ Fiji software and normalised optical density was presented as a mean+/−SD. Statistical analysis was performed using two-tailed, unpaired Student's t-test with GraphPad Prism 8 software, n=3.

### EXAMPLES

### Abbreviations

CDX101: N-(2-aminophenyl)-4-(1-[(1,3-dimethyl-1H-pyrazol-4-yl)methyl]piperidin-4-yl)benzamide
MHC: Major histocompatibility complex

### Example 1

### Cell Culture and Compound Treatment

Human colorectal adenocarcinoma HCT116 (ATCC® CCL-247; RRID:CVCL_0291), human lung cancer A549 (ATCC® CCL-185; RRID:CVCL JK07), two normal human bronchial epithelium BEAS-2B (ATCC® CRL-9609; RRID:CVCL_0168) and NBE1 (RRID:CVCL_9Y83), were cultured in Dulbecco's modified Eagle medium (DMEM) (Sigma-Aldrich, St. Louis, MO, USA) supplemented with 10% fetal bovine serum (Labtech, Heathfield, UK) and 1% penicillin/streptomycin (Gibco, Life Technologies, Carlsbad, CA, USA). All cell lines were tested for Mycoplasma contamination before use. CXD101 was dissolved in DMSO and used as described.

### RT-qPCR

RNA was isolated from cells using TRIzol (Thermo Fisher Scientific, Waltham, MA, USA) or the Direct-zol RNA MiniPrep kit (Zymo Research, Irvine, CA, USA) according to the manufacturer's instructions. 1 μg of total RNA was used for complementary DNA (cDNA) synthesis. Reverse transcription with oligo(dT)20 primer (Invitrogen, Carlsbad, CA, USA) was performed using SuperScript III Reverse Transcriptase (Invitrogen) as per the manufacturer's instructions. Quantitative reverse transcription PCR (qRT-PCR) was carried out in technical triplicate using the indicated primer pairs and the Brilliant Ill Ultra-Fast SYBR® Green QPCR Master Mix (Agilent, Santa Clara, CA, USA) on an AriaMX real-time qPCR instrument (Agilent). Results were expressed as average (mean) fold change compared to control treatments using the ΔΔCt method from three biological repeat experiments. Glyceraldehyde-phosphate dehydrogenase (GAPDH) primer sets were used as an internal calibrator. Error bars represent SE unless otherwise indicated.

### Western Blotting

Cell pellets were lysed in radio-immunoprecipitation assay buffer [50 mM tris-HCl (pH 8), 150 mM NaCl, 1% Igepal CA-630, 0.5% sodium deoxycholate, 0.1% SDS, 0.2 mM sodium orthovanadate, and protease inhibitor cocktails], for 30 minutes on ice and centrifuged for another 30 minutes at maximum speed at 4° C. Protein concentration was assessed by Bradford assay (Quick Start™ Bradford 1× Dye Reagent, Bio-Rad Laboratories, Hercules, CA, USA). After gel electrophoresis, proteins were transferred onto the PVDF or nitrocellulose membrane by means of Trans-Blot® Turbo™ Transfer System (Bio-Rad Laboratories) and blocked by 1h incubation in 5% skimmed milk (Merck Group, Darmstadt, Germany) in PBST at room temperature. The following antibodies were used in immunoblotting: anti-H3AcK9 (ab10812, Abcam; RRID:AB_297491), anti-H3AcK14 (#7627, Cell Signaling, Danvers, MA, USA; RRID:AB_10839410), anti-p-Actin (#3700, Cell Signaling; RRID:AB_2242334), all overnight at 4° C. Next, membranes were washed and treated with secondary antibody for 1 h at RT. Chemiluminescent signals were detected by LICOR C-Digit (LI-COR Biosciences, Lincoln, NE, USA), and the data quantified using ImageJ software (National Institutes of Health, Bethesda, MD, USA) (RRID:SCR_003070).

### Immunogenicity Experiment

Twenty C57BL/6 female mice (5 mice per group: control; CXD101 treated; peptide vaccinated; CXD101/peptide vaccinated) where 10 mice were vaccinated intravenously with 30 μg of peptides encoding CD8 T cell epitopes from the beta-galactosidase gene, βgale₉₆₋₁₀₃ (DAPIYTNV; D8V), the βgal₄₉₇₋₅₀₄ (ICPMYARV; 18V) (Genscript) with 30 μg polylC (InVivogen) and 25 μg anti-CD40 (2bScientific). 1-day post vaccination, half the mice were administered CXD101 (25 mg/kg) by oral gavage once a day for 5 days. Mice were boosted at day 7 with peptides in the presence of polylC and anti-CD40 as before. One day post-boost, the mice were again treated with CXD101 (25 mg/kg) once a day for 5 days. Mice were bled at day 6 and 13 post prime and the levels of peptide-specific CD8 T cells measured by detection with tetramers followed by flow cytometry.

### Immuno-Histochemistry

Lung tissues were harvested at day 14, fixed, and embedded in paraffin blocks and cut into 5 μm sections. FFPE slides were washed for 5 min with Histochoice (Sigma Aldrich), followed by two times 3 min washing in 100% Ethanol, 3 min in 70% Ethanol and 5 min in tap water. Next, samples were incubated with antigen retrieval solution (e.g. sodium citrate buffer or Tris/EDTA—depending on used antibody) at 99° C. in water bath for 20 min. After 3× washing with purified water, samples were incubated in freshly made 6% Methanol/H₂O₂ for 15 min and washed in tap water. In the next steps, slides were washed in 1% PBST for 5 min, blocked in blocking serum solution (Vectastatin ABC kit, Vector Laboratories, Burlingame, CA, USA) for 20 min., washed again in 1% PBST for 5 min and incubated overnight at 4° C. with primary antibody: anti-H3AcK9 (ab10812, Abcam; RRID:AB_297491). Samples were further stained with secondary antibody (Vectastain ABC kit) at room temperature. In the next step, ABC solution (VECTASTAIN® ABC-HRP Kit, Peroxidase, Rabbit IgG, PK-4001, Vector Laboratories) was added for 30 min, slides were washed in 1% PBST and incubated with DAB solution (Vector DAB) for another 10 min. Sections were counterstained with hematoxylin (Sigma Aldrich). Results were analysed using Leica DM2500 optical microscope and presented as semi-quantitative using ImageJ software (National Institutes of Health, USA).

### REFERENCES

(1) West A C & Johnstone R W (2014) New and emerging HDAC inhibitors for cancer treatment. J Clin Invest 124, 30-39, doi: 10.1172/JC169738.
(2) Cappellacci L, Perinelli D R, Maggi F, Grifantini M & Petrelli R (2020) Recent Progress in Histone Deacetylase Inhibitors as Anticancer Agents. Curr Med Chem 27, 2449-2493, doi: 10.2174/0929867325666181016163110.
(3) Hogg S J, Beavis P A, Dawson M A & Johnstone R W (2020) Targeting the epigenetic regulation of antitumour immunity. Nat Rev Drug Discov, doi: 10.1038/s41573-020-0077-5.
(4) Eyre T A, Collins G P, Gupta A, Coupe N, Sheikh S, Whittaker J, Wang L M, Campo L, Soilleux E, Tysoe F, Cousins R, La Thangue N, Folkes L K, Stratford M R L, Kerr D & Middleton MR (2019) A phase 1 study to assess the safety, tolerability, and pharmacokinetics of CXD101 in patients with advanced cancer. Cancer 125, 99-108, doi: 10.1002/cncr.31791.
(5) Fang H, Knezevic B, Burnham K L & Knight J C (2016) XGR software for enhanced interpretation of genomic summary data, illustrated by application to immunological traits. Genome Med 8, doi: ARTN 129 10.1186/s13073-016-0384-y.
(6) Love M A S, Huber W, 2019. DESeq2: Differential gene expression analysis based on the negative binomial distribution., Bioconductor version: Release (39).
(7) Hori S, Nomura T & Sakaguchi S (2017) Pillars Article: Control of Regulatory T Cell Development by the Transcription Factor Foxp3. Science 2003. 299: 1057-1061. J Immunol 198, 981-985.
(8) Cassetta L & Pollard J W (2018) Targeting macrophages: therapeutic approaches in cancer. Nat Rev Drug Discov 17, 887-904, doi: 10.1038/nrd.2018.169.
(9) Bertani F R, Mozetic P, Fioramonti M, luliani M, Ribelli G, Pantano F, Santini D, Tonini G, Trombetta M, Businaro L, Selci S & Rainer A (2017) Classification of M1/M2-polarized human macrophages by label-free hyperspectral reflectance confocal microscopy and multivariate analysis. Sci Rep 7, 8965, doi: 10.1038/s41598-017-08121-8.
(10) Jelencic V, Sestan M, Kavazovic I, Lenartic M, Marinovic S, Holmes T D, Prchal-Murphy M, Lisnic B, Sexl V, Bryceson Y T, Wensveen F M & Polic B (2018) NK cell receptor NKG2D sets activation threshold for the NCR1 receptor early in NK cell development. Nat Immunol 19, 1083-+, doi: 10.1038/s41590-018-0209-9.
(11) Havel J J, Chowell D & Chan T A (2019) The evolving landscape of biomarkers for checkpoint inhibitor immunotherapy. Nat Rev Cancer 19, 133-150, doi: 10.1038/s41568-019-0116-x.
(12) Wilkinson R W & Leishman AJ (2018) Further Advances in Cancer immunotherapy: Going Beyond Checkpoint Blockade. Front Immunol 9, doi: ARTN 108210.3389/fimmu.2018.01082.
(13) Castle J C, Loewer M, Boegel S, de Graaf J, Bender C, Tadmor A D, Boisguerin V, Bukur T, Sorn P, Paret C, Diken M, Kreiter S, Tureci O & Sahin U (2014) Immunomic, genomic and transcriptomic characterization of CT26 colorectal carcinoma. Bmc Genomics 15, doi: Artn 19010.1186/1471-2164-15-190.
(14) Varade J, Magadan S & Gonzalez-Fernandez A (2020) Human immunology and immunotherapy: main achievements and challenges. Cell Mol Immunol, doi: 10.1038/s41423-020-00530-6.
(15) Jin M Z & Jin W L (2020) The updated landscape of tumor microenvironment and drug repurposing. Signal Transduct Tar 5, doi: ARTN 16610.1038/s41392-020-00280-x.
(16) Tay R E, Richardson E K & Toh H C (2020) Revisiting the role of CD4(+)T cells in cancer immunotherapy-new insights into old paradigms. Cancer Gene Ther, doi: 10.1038/s41417-020-0183-x.
(17) Barnes T A & Amir E (2017) HYPE or HOPE: the prognostic value of infiltrating immune cells in cancer. Brit J Cancer 117, 451-460, doi: 10.1038/bjc.2017.220.
(18) Walker L S K (2013) Treg and CTLA-4: Two intertwining pathways to immune tolerance. J Autoimmun 45, 49-57, doi: 10.1016/j.jaut.2013.06.006.
(19) Gaudino S J & Kumar P (2019) Cross-Talk Between Antigen Presenting Cells and T Cells Impacts Intestinal Homeostasis, Bacterial Infections, and Tumorigenesis. Front Immunol 10, doi: ARTN 36010.3389/fimmu.2019.00360.
(20) Wherry E J (2011) T cell exhaustion. Nat Immunol 12, 492-499, doi: 10.1038/ni.2035.
(21) Jenkins R W, Barbie D A & Flaherty K T (2018) Mechanisms of resistance to immune checkpoint inhibitors. Brit J Cancer 118, 9-16, doi: 10.1038/bjc.2017.434.
(22) Vinay D S, Ryan E P, Pawelec G, Talib W H, Stagg J, Elkord E, Lichtor T, Decker W K, Whelan R L, Kumara HMCS, Signori E, Honoki K, Georgakilas A G, Amin A, Helferich W G, Boosani C S, Guha G, Ciriolo M R, Chen S, Mohammed S I, Azmi A S, Keith W N, Bilsland A, Bhakta D, Halicka D, Fujii H, Aquilano K, Ashraf S S, Nowsheen S, Yang X J, Choi B K & Kwon B S (2015) Immune evasion in cancer: Mechanistic basis and therapeutic strategies. Semin Cancer Biol 35, S185-S198, doi: 10.1016/j.semcancer.2015.03.004.
(23) He X & Xu C Q (2020) Immune checkpoint signaling and cancer immunotherapy. Cell Res 30, 660-669, doi: 10.1038/s41422-020-0343-4.
(24) Alsaab H O, Sau S, Alzhrani R, Tatiparti K, Bhise K, Kashaw S K & lyer A K (2017) PD-1 and PD-L1 Checkpoint Signaling Inhibition for Cancer Immunotherapy: Mechanism, Combinations, and Clinical Outcome. Front Pharmacol 8, doi: ARTN 56110.3389/fphar.2017.00561.
(25) Oliveira A F, Bretes L & Furtado I (2019) Review of PD-1/PD-L1 Inhibitors in Metastatic dMMR/MSI-H Colorectal Cancer. Front Oncol 9, doi: ARTN 39610.3389/fonc.2019.00396.
(26) Orillion A, Hashimoto A, Damayanti N, Shen L, Adelaiye-Ogala R, Arisa S, Chintala S, Ordentlich P, Kao C A, Elzey B, Gabrilovich D & Pili R (2017) Entinostat Neutralizes Myeloid-Derived Suppressor Cells and Enhances the Antitumor Effect of PD-1 Inhibition in Murine Models of Lung and Renal Cell Carcinoma. Clinical Cancer Research 23, 5187-5201, doi: 10.1158/1078-0432.Ccr-17-0741.
(27) Wang X G, Waschke B C, Woolaver R A, Chen Z G, Zhang G, Piscopio A D, Liu X D & Wang J H (2019) Histone Deacetylase Inhibition Sensitizes PD1 Blockade-Resistant B-cell Lymphomas. Cancer Immunology Research 7, 1318-1331, doi: 10.1158/2326-6066.Cir-18-0875.
(28) Woods D M, Sodre A L, Villagra A, Sarnaik A, Sotomayor E M & Weber J (2015) HDAC Inhibition Upregulates PD-1 Ligands in Melanoma and Augments Immunotherapy with PD-1 Blockade. Cancer Immunology Research 3, 1375-1385, doi: 10.1158/2326-6066.Cir-15-0077-T.
(29) Zheng H, Zhao W P, Yan C H, Watson C C, Massengill M, Xie M Y, Massengill C, Noyes D R, Martinez G V, Afzal R, Chen Z H, Ren X B, Antonia S J, Haura E B, Ruffell B & Beg A A (2016) HDAC Inhibitors Enhance T-Cell Chemokine Expression and Augment Response to PD-1 Immunotherapy in Lung Adenocarcinoma. Clinical Cancer Research 22, 4119-4132, doi: 10.1158/1078-0432.Ccr-15-2584.
(30) Burke B, Eden C, Perez C, Belshoff A, Hart S, Plaza-Rojas L, Delos Reyes M, Prajapati K, Voelkel-Johnson C, Henry E, Gupta G & Guevara-Patino J (2020) Inhibition of Histone Deacetylase (HDAC) Enhances Checkpoint Blockade Efficacy by Rendering Bladder Cancer Cells Visible for T Cell-Mediated Destruction. Front Oncol 10, doi: ARTN 69910.3389/fonc.2020.00699.
(31) Smith H J, McCaw T R, Londono A I, Katre A A, Meza-Perez S, Yang E S, Forero A, Buchsbaum D J, Randall T D, Straughn J M, Norian L A & Arend R C (2018) The antitumor effects of entinostat in ovarian cancer require adaptive immunity. Cancer 124, 4657-4666, doi: 10.1002/cncr.31761.
(32) West A C, Smyth M J & Johnstone R W (2014) The anticancer effects of HDAC inhibitors require the immune system. Oncoimmunology 3, doi: ARTN e2741410.4161/onci.27414.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in anyway.

The use of any and all examples, or exemplary language (e.g., “such as”) provided herein, is intended merely to better illuminate the invention and does not pose a limitation on the scope of the invention unless otherwise paragraphed. No language in the specification should be construed as indicating any non-paragraphed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the paragraphs appended hereto as permitted by applicable law.

1. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for use as a vaccine adjuvant.

2. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, according to claim 1, for use as an immune potentiating agent.

3. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, according to claim 1, for use as an immune sensitising agent.

4. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, according to claims 1 to 3, for use in a method of potentiating an immune response in a subject in need thereof, the method comprising administering an effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

5. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, according to claim 4, wherein the immune response is induced or enhanced.

6. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof according to claims 4 or 5 wherein the subject is susceptible to or may have a disease, disorder or condition to which an immune response is required.

7. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof according to claims 4 to 6, wherein the subject is susceptible to or may have a pre-cancerous condition, cancer, an infectious disease, or an allergy.

8. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, according to claims 1 to 7, for use in the treatment or prevention of a disease, disorder or condition to which an immune response is required, wherein the N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered in combination with a therapeutically effective amount of a vaccine.

9. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, according to claims 1 to 8 wherein N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered simultaneously, separately or sequentially with a vaccine.

10. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for the use as claimed in claims 8 or 9, wherein the vaccine is an allergen vaccine, an infectious disease vaccine, a mucosal vaccine or an anti-cancer vaccine.

11. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for the use as claimed in claim 10, wherein the infectious disease vaccine is a vaccine used to treat or prevent viral, bacterial, fungal, protozoa and/or parasite infections.

12. N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, for the use in any one of the claims 1 to 11, wherein N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered at a dosage of 0.1 mg to 40 mg per day.

13. A method of treating or preventing a disease, disorder, condition to which an immune response is required, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

14. A method of treating or preventing a disease, disorder, condition to which an immune response is required, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

15. A method of potentiating an immune response to a vaccine in a subject, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

16. A method of potentiating the effect of a vaccine, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

17. A method of sensitising the immune system of a subject, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, to a subject receiving vaccine therapy.

18. A method of sensitising the immune system of a subject, the method comprising administering to a subject in need thereof a therapeutically effective amount of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a therapeutically effective amount of a vaccine.

19. A method according to claims 13 to 18 wherein N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered simultaneously, separately or sequentially with a vaccine.

20. Use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use in the treatment of a disease, disorder or condition to which an immune response is required.

21. Use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use as a vaccine adjuvant.

22. Use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use as an immune-sensitiser.

23. Use of N-(2-aminophenyl)-4-(1-[(1,3-dimethyl-1H-pyrazol-4-yl)methyl]piperidin-4-yl)benzamide (CXD101/zabinostat), or a pharmaceutically acceptable salt thereof, in the manufacture of a medicament for use as an immune-potentiating agent.

24. A combination comprising N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, and a vaccine.

25. A combination according to claim 24 wherein the vaccine is an allergen vaccine, an infectious disease vaccine, a mucosal vaccine or an anti-cancer vaccine.

26. A combination according to claims 24 and 25 wherein N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered simultaneously, separately or sequentially with the vaccine.

27. A combination according to claims 24 to 26 wherein N-(2-aminophenyl)-4-(1-[(1,3-dimethyl-1H-pyrazol-4-yl)methyl]piperidin-4-yl)benzamide (CXD101/zabinostat), or a pharmaceutically acceptable salt or solvate thereof, is administered at dosage of 0.1 mg to 40 mg per day.

28. A pharmaceutical product comprising a combination according to claims 24 to 27.

29. A pharmaceutical composition comprising a combination according to claims 24 to 27, and one or more pharmaceutically acceptable excipients.

30. A combination according to any one claims 24 to 27, or a pharmaceutical product according to claim 28, or a pharmaceutical composition according to claim 29 for use in therapy.

31. A combination, a pharmaceutical product, or a pharmaceutical composition according to claim 30 for use in the treatment or prevention of disease, disorder or condition to which an immune response is required in a subject.

32. A combination, a pharmaceutical product, or a pharmaceutical composition according to claims 30 and 31 for use in the treatment or prevention of an allergy, infectious disease, a pre-cancerous condition, or cancer.

33. A combination, a pharmaceutical product, or a pharmaceutical composition according to claim 32 wherein the infectious disease is a viral infectious disease or a bacterial infectious disease.

34. A method of potentiating an immune response in a subject, the method comprising administering a therapeutically effective amount of the combination according to claims 24 to 27, a therapeutically effective amount of the pharmaceutical product according to claim 28, or a therapeutically effective amount of the pharmaceutical composition according to claim 29 to the subject in need thereof.

35. A method of potentiating the immune response to a vaccine in a subject, the method comprising administering a therapeutically effective amount of the combination according to claims 24 to 27, a therapeutically effective amount of the pharmaceutical product according to claim 28, or a therapeutically effective amount of the pharmaceutical composition according to claim 29 to the subject in need thereof.

36. A method of sensitising the immune system of a subject, the method comprising administering a therapeutically effective amount the combination according to claims 24 to 27, a therapeutically effective amount of the pharmaceutical product according to claim 28, or a therapeutically effective amount of the pharmaceutical composition according to claim 29 to the subject in need thereof.

37. A method of preventing or treating a disease, disorder or condition to which an immune response is required in a subject, the method comprising administering a therapeutically effective amount the combination according to claims 24 to 27, a therapeutically effective amount of the pharmaceutical product according to claim 28, or a therapeutically effective amount of the pharmaceutical composition according to claim 29 to the subject in need thereof.

38. Use of a combination according to claims 24 to 27 in the manufacture of a medicament for treating or preventing a disease, disorder or condition to which an immune response is required in a subject.
